(19) United States
(12) Patent Application Publication
(10) Pub. No.: US 2014/0255402 A1
(43) Pub. Date: Sep. 11, 2014

(54) METHOD OF TREATING LEUKEMIA IN A MAMMAL

(71) Applicants: Amit Verma, Bronxville, NY (US); Stefan Klaus Barta, Long Island City, NY (US)

(72) Inventors: Amit Verma, Bronxville, NY (US); Stefan Klaus Barta, Long Island City, NY (US)

(73) Assignee: ALBERT EINSTEIN COLLEGE OF MEDICINE OF YESHIVA UNIVERSITY, Bronx, NY (US)

(21) Appl. No.: 14/190,705

(22) Filed: Feb. 26, 2014

Related U.S. Application Data
(60) Provisional application No. 61/774,772, filed on Mar. 8, 2013.

(51) Int. Cl. A61K 39/395; A61K 31/7068

(52) U.S. Cl. CPC A61K 39/3955; A61K 31/7068; A61K 2039/507
USPC 424/134.1; 424/130.1; 424/133.1

(57) ABSTRACT

The present invention provides a method of treating a leukemia in a mammal comprising sequentially administering to the mammal an amount of an immunotoxin and an amount of a chemotherapeutic agent effective to treat the leukemia.

### CROSS-REFERENCE TO RELATED APPLICATIONS

This application claims benefit of U.S. Provisional Application No. 61/774,772, filed Mar. 8, 2013, the contents of which are hereby incorporated by reference.

### BACKGROUND OF THE INVENTION

Throughout this application, various publications are referred to in square brackets. Full citations for these references may be found at the end of the specification. The disclosures of these publications, and all patents, patent application publications and books referred to herein are hereby incorporated by reference in their entirety into the subject application to more fully describe the art to which the subject invention pertains.

Leukemia is often resistant to treatment. Relapsed or refractory (r/r) acute lymphoblastic leukemia (ALL) in adults has a poor prognosis when treated with conventional therapy. Only 7-12% of these patients become long-term survivors [1,2]. Novel approaches are urgently needed to improve the outcomes for this patient population. One promising new approach is exemplified by a class of anticancer agents termed immunoconjugates (ICs) or immunotoxins (ITs). Immunotoxins are composed of a potent toxin linked to a moiety designed to specifically target tumor cells [3,4]. These agents have two main advantages over cytotoxic agents: firstly, by directly targeting certain surface markers they maximize drug delivery to tumor cells without increasing toxicity to normal cells; secondly, they use a different mechanism to induce cell killing when compared with traditional cytotoxic drugs, thus potentially circumventing chemoresistance.

Combotox is such an IT. It is a 1:1 mixture of two murine monoclonal immunoglobulin G1 (IgG1) antibodies: HD37, directed against CD19; and RFB4, directed against CD22. They are coupled to a deglycosylated ricin-A chain (dgRTA) [5]. RTA inhibits protein synthesis by catalytically inactivating the ribosomal RNA. The antigens targeted with this drug, CD 19 and CD22, are present on leukemic blasts in the vast majority of patients with B-lineage ALL [6,7]. Immunoconjugates targeting CD19, CD22, or both antigens at the same time, have shown promising efficacy in r/r ALL in early clinical trials [8-13]. Although HD37-dgRTA and RFB4-dgRTA are active when given individually, their ability to kill tumor cells is additive when both are given together, and the combination is more efficacious than either agent alone in vivo [14].

The safety and efficacy of Combotox has been explored in two phase I clinical trials: one in pediatric patients and another in adult patients, both with r/r ALL [8,13]. Single-agent Combotox proved efficacious with an overall response rate (complete, partial and hematological response) of 53% for children and 31% for adults, but the responses were usually short-lived.

The present invention addresses the need for improved leukemia treatments by combining conventional chemotherapy with immunotoxins.

### SUMMARY OF THE INVENTION

The present invention addresses a need for more effective methods of treating leukemia in a mammal. The present invention is a method of treating the leukemia comprising sequentially administering to the mammal an amount of immunotoxin and an amount of chemotherapeutic agent effective to treat the leukemia.

### BRIEF DESCRIPTION OF THE DRAWINGS

FIGS. 1A-1B: FIG. 1 shows lymphoblasts (black arrows) in the peripheral blood of a NOD mouse injected with NALM/6 cells on day 7 (panel A) and day 14 (panel B) after inoculation.

FIGS. 2A-2C: FIG. 2 shows several possible schedules for administering cytarabine and Combotox. FIG. 2A is a schema showing an administration schedule for concurrent administration of cytarabine and Combotox. FIG. 2B is a schema showing an administration schedule for sequential administration of cytarabine and Combotox. FIG. 2C is a schema showing an administration schedule for a comparison of concurrent and sequential administration of cytarabine and Combotox.

FIGS. 3A-3C: FIG. 3A shows the survival times of mice after concurrent administration of cytarabine and Combotox. FIG. 3B shows the survival times of mice after sequential administration of cytarabine and Combotox. FIG. 3C is a comparison of the respective survival times of mice treated with concurrent administration of cytarabine and Combotox and mice treated with sequential administration of cytarabine and Combotox.

### DETAILED DESCRIPTION OF THE INVENTION

The present invention provides a method of treating a leukemia in a mammal comprising sequentially administering to the mammal an amount of immunotoxin and an amount of chemotherapeutic agent effective to treat a leukemia.

In an embodiment, the leukemia may comprise relapsed acute lymphoblastic leukemia or refractory acute lymphoblastic leukemia. In an embodiment, the mammal is a human.

In a preferred embodiment, the chemotherapeutic agent is an agent that interferes with DNA synthesis. In an embodiment, the chemotherapeutic agent comprises cytarabine.

By way of illustration but not limitation, chemotherapeutic agents as used in the invention may include alkylating agents, such as cyclophosphamide, mechlorethamine, chlorambucil, and melphalan; anthracyclines, such as daunorubicin, doxorubicin, epirubicin, idarubicin, mitoxantrone, and valrubicin; cytoskeletal disruptors, such as paclitaxel and docetaxel; inhibitors of topisomerase I, such as irinotecan and topotecan; inhibitors of topoisomerase II, such as etoposide, teniposide, and tafluposide; kinase inhibitors, such as bortezomib, erolitinib, gefitinib, imatinib, venumafenib, and vismodegib; monoclonal antibodies, such as bevacizumab, cetuximab, ipilimumab, ofatumumab, ocrelizumab, panitumab, and rituximab; nucleotide analogs and nucleotide-precursor analogs, such as azacitidine, azathioprine, capecitabine, cytarabine, doxifluridine, fluorouracil, gemcitabine, hydroxyurea, mercaptopurine, methotrexate, and tioguanine; peptide antibiotics, such as bleomycin and actinomycin; platinum-based agents, such as carboplatin, cisplatin, and oxaliplatin; retinoids, such as tretinoin, alitretinoin, and bexarotene; vinca alkaloids and derivatives, such as vinblastine, vincristine, vindesine, and vinorelbine; and epothilones.

In a preferred embodiment, the immunotoxin comprises a combination of (1) an anti-CD19 antibody or CD19-binding fragment thereof and (2) an anti-CD22 antibody or a CD22-binding fragment thereof. By way of illustration but not limitation, immunotoxins may include such antibodies, each conjugated or otherwise bound a toxin.

In an embodiment, the antibodies are, independently, chosen from murine antibodies, human antibodies, humanized antibodies or chimeric antibodies. In an embodiment, the antibodies are, independently, monoclonal antibodies. In an embodiment, the antibodies are, independently, IgG antibodies. In an embodiment, the antibodies are, independently, IgG1 antibodies. As used herein, the term “antibody” refers to an intact antibody, i.e. with complete Fc and Fv regions. “Fragment” refers to any portion of an antibody, or portions of an antibody linked together, such as, in non-limiting examples, a Fab, F(ab)2, a single-chain Fv (scFv), which is less than the whole antibody but which is an antigen-binding portion and which competes with the intact antibody of which it is a fragment for specific binding. As such a fragment can be prepared, for example, by cleaving an intact antibody or by recombinant means. See generally, Fundamental Immunology, Ch. 7 (Paul, W., ed., 2nd ed. Raven Press, N.Y. (1989), hereby incorporated by reference in its entirety). Antigen-binding fragments may be produced by recombinant DNA techniques or by enzymatic or chemical cleavage of intact antibodies or by molecular biology techniques. In some embodiments, a fragment is an Fab, Fab′, F(ab′)2, Fd , Fv, complementarity determining region (CDR) fragment, single-chain antibody (scFv), (a variable domain light chain (VL) and a variable domain heavy chain (VH) linked via a peptide linker.

In an embodiment, the antibodies are monoclonal antibodies. The term “monoclonal antibody” as used herein refers to an antibody member of a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies.

In an embodiment of the inventions described herein, the antibody is isolated. As used herein, the term “isolated antibody” refers to an antibody that by virtue of its origin or source of derivation has one, two, three or four of the following: (1) is not associated with naturally associated components that accompany it in its native state, (2) is free of other proteins from the same species, (3) is expressed by a cell from a different species, and (4) does not occur in nature.

In an embodiment the composition or pharmaceutical composition comprising one or more of the antibodies or fragments described herein is substantially pure with regard to the antibody or fragment. A composition or pharmaceutical composition comprising one or more of the antibodies or fragments described herein is “substantially pure” with regard to the antibody or fragment when at least about 60 to 75% of a sample of the composition or pharmaceutical composition exhibits a single species of the antibody or fragment. A substantially pure composition or pharmaceutical composition comprising one or more of the antibodies or fragments described herein can comprise, in the portion thereof which is the antibody or fragment, 60%, 70%, 80% or 90% of the antibody or fragment of the single species, more usually about 95%, and preferably over 99%. Antibody purity or homogeneity may tested by a number of means well known in the art, such as polyacrylamide gel electrophoresis or HPLC.

As used herein, a “human antibody” unless otherwise indicated is one whose sequences correspond to (i.e. are identical in sequence to) an antibody that could be produced by a human and/or has been made using any of the techniques for making human antibodies as disclosed herein, but not one which has been made in a human. This definition of a human antibody specifically excludes a humanized antibody. A “human antibody” as used herein can be produced using various techniques known in the art, including phage-display libraries (e.g. Hoogenboom and Winter, J. Mol. Biol., 227:381 (1991); Marks et al., J. Mol. Biol., 222:581 (1991), hereby incorporated by reference in its entirety), by methods described in Cole et al., Monoclonal Antibodies and Cancer Therapy, Alan R. Liss, p. 77 (1985) (hereby incorporated by reference in its entirety); Boerner et al., J. Immunol., 147(1):86-95 (1991) (hereby incorporated by reference in its entirety), van Dijk and van de Winkel, Curr. Opin. Pharmacol., 5: 368-74 (2001) (hereby incorporated by reference in its entirety), and by administering the antigen (e.g. CD19) to a transgenic animal that has been modified to produce such antibodies in response to antigenic challenge, but whose endogenous loci have been disabled, e.g., immunized xenomice (see, e.g., U.S. Pat. Nos. 5,939,598; 6,075,181; 6,114,598; 6,150,584 and 6,162,963 to Kucherlapati et al. regarding XENOMOUSE™ technology, each of which patents are hereby incorporated by reference in their entirety), e.g. Veloclmmune® (Regeneron, Tarrytown, N.Y.), e.g. UltiMab® platform (Medarex, now Bristol Myers Squibb, Princeton, N.J.). See also, for example, Li et al., Proc. Natl. Acad. Sci. USA, 103:3557-3562 (2006) regarding human antibodies generated via a human B-cell hybridoma technology. See also KM Mouse® system, described in PCT Publication WO 02/43478 by Ishida et al., in which the mouse carries a human heavy chain transchromosome and a human light chain transgene, and the TC mouse system, described in Tomizuka et al. (2000) Proc. Natl. Acad. Sci. USA 97:722-727, in which the mouse carries both a human heavy chain transchromosome and a human light chain transchromosome, both of which are hereby incorporated by reference in their entirety. In each of these systems, the transgenes and/or transchromosomes carried by the mice comprise human immunoglobulin variable and constant region sequences.

The term “human antibody”, as used herein, is intended to include antibodies having variable regions in which both the framework and CDR regions are sequences of human origin or identical thereto other than antibodies naturally occurring in a human or made in a human. Furthermore, if the antibody (e.g. an intact antibody rather than, for example, an Fab fragment) contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. In one non-limiting embodiment, where the human antibodies are human monoclonal antibodies, such antibodies can be produced by a hybridoma which includes a B cell obtained from a transgenic nonhuman animal, e.g., a transgenic mouse, having a genome comprising a human heavy chain transgene and a light chain transgene fused to an immortalized cell.

In an embodiment, the chemotherapeutic agent may be administered at a dosage of 100-300 milligrams per square meter of the mammal's skin area. In an embodiment, the chemotherapeutic agent may be administered at a dosage of about 200 milligrams per kilogram of body weight.

In an embodiment, the chemotherapeutic is cytarabine. In an embodiment, the cytarabine is administered to a human at a dosage of 50-3000 milligrams per square meter of the human's skin area.

In an embodiment, the immunotoxin comprises a combination of anti-CD19 and anti-CD22 antibodies, where the anti-CD19 antibody is administered at a dosage of 1-4 milligrams per kilogram of body weight and the anti-CD22 antibody is administered at a dosage of 0.5-2.5 milligrams per kilogram of body weight. In an embodiment, the immunotoxin comprises a combination of anti-CD19 and anti-CD22 antibodies, where the anti-CD19 antibody is administered at a dosage of 0.5-1.5 milligrams per kilogram of body weight and the anti-CD22 antibody is administered at a dosage of 0.1-1.0 milligrams per kilogram of body weight.

In an embodiment, the immunotoxin comprises an anti-CD19 antibody and an anti-CD22 antibody, each independently coupled to deglycosylated ricin-A. In an embodiment, the anti-CD19 and/or anti-CD22 antibodies are monoclonal antibodies. In an embodiment, the anti-CD19 and/or anti-CD22 antibodies are isolated human antibodies, isolated humanized antibodies, or isolated chimeric antibodies. In an embodiment, the anti-CD19 antibody comprises the epitope-binding portion of an HD37 antibody, and the anti-CD22 antibody comprises the epitope-binding portion of an RFB4 antibody. In a further embodiment, such antibodies are chimeric or humanized.

In one embodiment, the immunotoxin is administered before the chemotherapeutic agent is administered. In another embodiment, the chemotherapeutic agent is administered before the immunotoxin is administered. In an embodiment, the immunotoxin and chemotherapeutic agent are each administered a plurality of times, but in a sequential fashion and are not concurrently or simultaneously administered. In a preferred embodiment, the sequential administration comprises treatment with the chemotherapeutic agent on three consecutive days; four subsequent days of no administration of the chemotherapeutic agent or the immunotoxin; treatment with immunotoxin on the next subsequent day; one subsequent day of no administration of the chemotherapeutic agent or the immunotoxin; treatment with immunotoxin on the next subsequent day; one subsequent day of no administration of the chemotherapeutic agent or the immunotoxin; and treatment with immunotoxin on the next subsequent day.

In an embodiment, the sequential administration comprises administration of the chemotherapeutic and subsequent administration of the immunotoxin. In an embodiment, the sequential administration comprises administration of the chemotherapeutic and subsequent administration of the immunotoxin and then a subsequent administration of the chemotherapeutic. In an embodiment, the sequential administration comprises administration of the immunotoxin and subsequent administration of the chemotherapeutic. In an embodiment, the sequential administration comprises administration of the immunotoxin and subsequent administration of the chemotherapeutic and then subsequent administration of the immunotoxin. In an embodiment, the immunotoxin is not administered within one of at least 1 hr., 2 hrs., 4 hrs., 8 hrs., 24 hrs or 48 hrs. of the administration of the chemotherapeutic.

In an embodiment, the leukemia is an advanced leukemia. In a further embodiment, the leukemia is characterized by the presence of peripheral blasts in the mammal. In an embodiment, the leukemia is refractory or relapsed.

“And/or” as used herein, for example, with option A and/or option B, encompasses the separate embodiments of (i) option A, (ii) option B, and (iii) option A plus option B.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Details

Summary: The outcome for patients with refractory or relapsed acute lymphoblastic leukemia (ALL) treated with conventional therapy is poor. Immunoconjugates present a novel approach and have recently been shown to have efficacy in this setting. Combotox is a mixture of two ricin-conjugated monoclonal antibodies (RFB4 and HD37) directed against CD19 and CD22, respectively, and has shown activity in pediatric and adult ALL. A murine xenograft model of advanced ALL was created, using the NALM/6 cell line to explore whether the combination of Combotox with the cytotoxic agent cytarabine (Ara-C) results in better outcomes. In this model, the combination of both low-dose and high-dose Combotox with Ara-C resulted in significantly longer median survival. Sequential administration of Ara-C and Combotox, however, was shown to be superior to concurrent administration of the same.

### Materials and Methods

Murine Xenograft Model: A human pre-B lymphoblast cell line NALM/6 [16] was cultured in RPMI 1640 medium with 15% fetal bovine serum and incubated at 37° C. with 5% CO₂ and 95% humidified air. The cells were maintained at a concentration of 4-9×10⁵ cells/mL by adding fresh medium daily. Before inoculation, the cells were concentrated by centrifugation to 50×10⁶ cells/mL and re-suspended in serum-free medium. Six-to-eight week old non-obese diabetic mice (NOD.Cg-Prkdc^{scid} I12rg^{tm1Wj1}/SzJ) were purchased from The Jackson Laboratory (Bar Harbor, Me.). These mice are maintained and used only under an animal use protocol approved by the Animal Institute Committee of Albert Einstein College of Medicine.

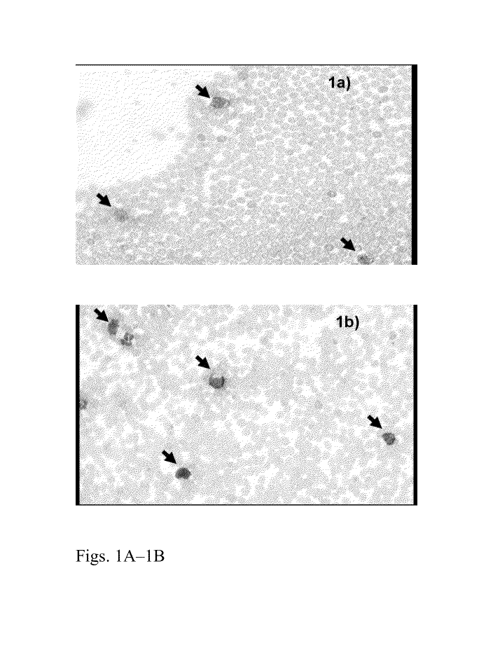

The mice were inoculated via tail vein injection with 8-10×10⁶ NALM/6 cells to establish a murine xenograft model of advanced B-lineage ALL. About 50 μL of blood from each mouse was taken from a facial vein on the day of administration of the first treatment. The presence of leukemic blasts in the blood was confirmed microscopically (FIG. 1).

Combotox: Combotox is a 1:1 mixture of anti-CD19 (HD37)-dgRTA and anti-CD22 (RFB4)-dgRTA. Each of murine monoclonal IgG1 antibody RFB4 (anti-CD22) and HD37 (anti-CD19) are each coupled to their own deglycosylated ricin-A chain (dgRTA) via a heterobifunctional, thiol-containing cross-linker, N-succinimidyl-oxycarbonyl-a-methyl-a-(2-pyridyldithio)toluene (SMPT) [5].

RFB4-dgRTA and HD37-dgRTA were prepared in the Good Manufacturing Practice (GMP) laboratory at the University of Texas Southwestern Medical Center (UTSWMC) and shipped overnight on dry ice. They were stored in a −85° C. freezer. On the day of planned administration, the vials were thawed and mixed in a 1:1 ratio to give Combotox. The infusions were held at 4° C. until administered.

Cytarabine: Cytarabine (or Ara-C) was purchased from Hospira (Hospira, Inc., Lake Forest, Ill.) as vials of 100 mg dry powder for injection. The powder was then reconstituted for injection on the day of administration with normal saline.

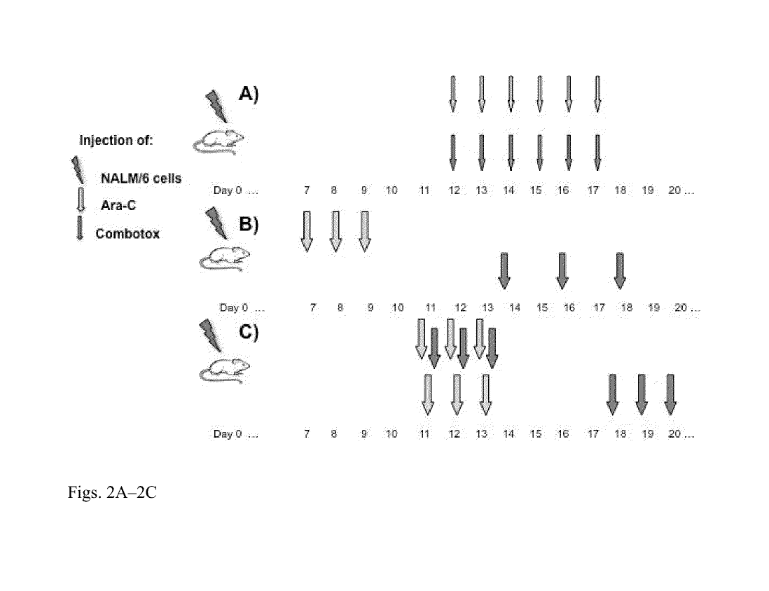

Treatment on a Concurrent Schedule: In the first experiment mice were divided into four groups and treated daily with intravenous injection of: (1) Ara-C, (2) Combotox, (3) Ara-C concurrent with Combotox or (4) normal saline (vehicle control), respectively. The doses were 100 mg/kg daily×6 for Ara-C and 0.8 mg/kg anti-CD19-dgRTA plus 0.5 mg/kg anti-CD22-dgRTA per injection daily×6 for Combotox (FIG. 2(A)). Treatment started on day 12, counted from the day of NALM/6 inoculation.

Treatment on a Sequential Schedule: In the second experiment, mice were also divided into four groups. The treatment was similar to the first experiment, except that the combination treatment with Ara-C and Combotox was on a sequential schedule. The treatment schedule was as follows: (1) Ara-C on days 7-9 after inoculation; (2) Combotox on days 14, 16 and 18; (3) Ara-C on days 7-9 followed by Combotox on days 14, 16 and 18; or (4) normal saline (control) on days 7-9, 14, 16 and 18. The doses were 200 mg/kg daily ×3 for Ara-C and 2.4 mg/kg anti-CD19-dgRTA plus 1.5 mg/kg anti-CD22-dgRTA per injection every other day×3 for Combotox (FIG. 2B).

Comparison of Concurrent and Sequential Schedules: In the third experiment, the combination treatment between the concurrent and sequential schedules were compared. The mice were randomly divided into three groups of eight mice each on day 11. Two groups were treated with tail vein injections of 200 mg/kg daily Ara-C for 3 days (days 11-13) and Combotox (2.4 mg/kg anti-CD19+1.5 mg/kg anti-CD22 daily ×3). Combotox was given either concurrently with Ara-C on days 11-13 (concurrent schedule) or sequentially on days 18-20 (sequential schedule). The third group of mice was injected intravenously with an equal volume of normal saline and served as vehicle control (FIG. 2C).

Statistics: The median survival time (MST) for each treatment group was calculated. Overall survival was plotted as a Kaplan-Meier curve and a two-sided log-rank test was performed. A p-value <0.05 indicated statistical significance. Animals that died immediately after injection of treatment agents (accidental deaths) were excluded from the analysis. The statistical analysis was performed using GraphPad Prism(R) version 5.0 (GraphPad Software, La Jolla, Calif.).

### Results

Inoculation of NALM/6 Cells into NOD Mice Results in an In Vivo Model of Advanced Disease: Since relapsed or refractory ALL usually presents in advanced stages with increased peripheral blasts, the goal was to develop an in vivo animal model that represented this stage of the human disease. Therefore, a large number of NALM/6 cells (8-10×10⁶) were injected in the tail veins of NOD mice. Peripheral smears of animals were tested at various days after inoculation. Numerous peripheral blood leukemic cells were detected from day 7 onward, mimicking advanced disease in humans. Thus, this was an adequate time to test the therapeutic potential of the antileukemic agents Combotox and cytarabine.

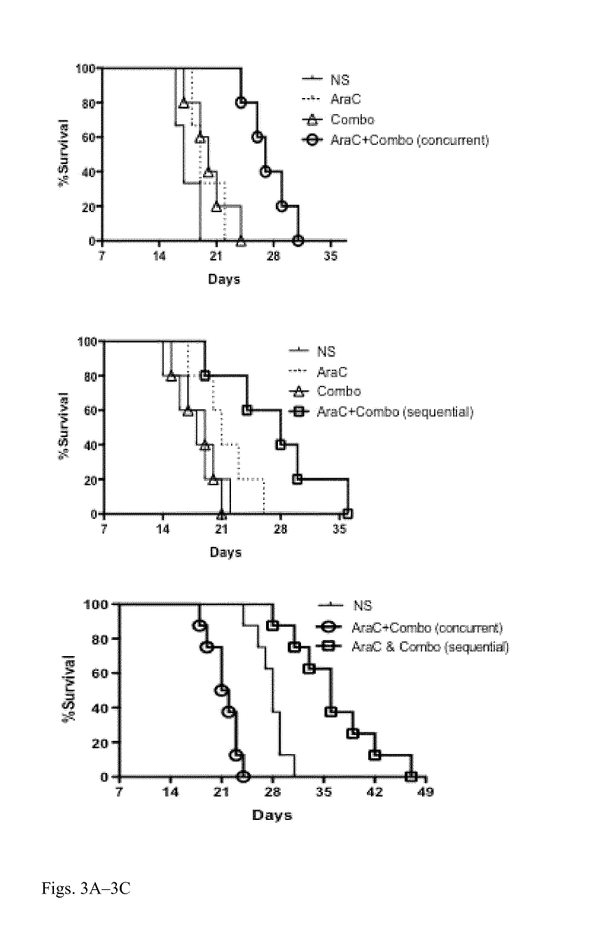

Concurrent Administration of Low Doses of Chemotherapy and Immunotherapy Leads to Increased Survival: The first cohort received 100 mg/m² of Ara-C given daily for 6 days and 0.8 mg/kg anti-CD19-dgRTA plus 0.5 mg/kg anti-CD22-dgRTA (Combotox) per injection daily for 6 days. These regimens were tested alone and concurrently with each other. The combination therapy led to increased survival, with a MST of 27 days in the cohort that received concurrent chemoimmunotherapy compared to 17, 19 and 20 days, respectively, in the placebo, Ara-C only and Combotox only treated groups (p-value <0.05; Table I and FIG. 3A).

TABLE I
Median survival times (MSTs) for each cohort of each experiment.

| Group | MST | p-Value* |
|---|---|---|
| Concurrent (experiment 1) | | |
| 1 (Ara-C; n = 5) | 19 | <0.05 |
| 2 (Combotox; n = 5) | 20 | |
| 3 (Ara-C and Combotox; n = 5) | 27 | |
| 4 (normal saline; n = 5) | 17 | |
| Sequential (experiment 2) | | |
| 1 (Ara-C; n = 5) | 20 | <0.05⁺ |
| 2 (Combotox; n = 5) | 18 | |
| 3 (Ara-C and Combotox; n = 5) | 28 | |
| 4 (normal saline; n = 5) | 19 | |
| Concurrent vs. sequential (experiment 3) | | |
| 1 (Ara-C and Combotox concurrent; n = 8) | 22 | <0.001 |
| 2 (Ara-C and Combotox sequential; n = 8) | 36 | |
| 3 (normal saline; n = 8) | 28 | |

*p-Value calculated for overall survival difference with two-sided log-rank test.
⁺p-Value for survival analysis calculated excluding mice that died accidentally on day of facial vein blood draw (two per group). If these mice were included, p-value would be 0.13.

Sequential Administration of High Doses Leads to Increased Survival: For the second and third experiments, the total dose of Combotox was increased to reflect the equivalence of human doses that have been used in the clinical setting [13]. The dose of Combotox was 2.4 mg/kg anti-CD19-dgRTA plus 1.5 mg/kg anti-CD22-dgRTA per injection×3, which was derived from calculation of the maximum tolerated dose (MTD) in humans. Additionally, the daily dose of Ara-C was increased to 200 mg/m² and treatment was given over a shorter period of time, thereby mimicking regimens used in the treatment of acute leukemias in humans.

In the second experiment these higher doses were tested alone or sequentially in mice with advanced disease. These treatments resulted in significantly improved MST in the combination group (MST 28 days) compared to 19, 20 and 18 days, respectively, in the placebo, Ara-C only and Combotox only treated groups (p-value <0.05; Table I and FIG. 3B).

Having demonstrated the efficacy of the low-dose concurrent combination and high-dose sequential combination, the subsequent experiment was a head-to-head comparison of these two regimens at the higher dose level. Thus, mice in the third experiment were treated by either Ara-C given concurrently with Combotox on days 11-13 or Ara-C given by itself on days 11-13 followed by Combotox on days 18-20. In this experiment only the sequential administration of Ara-C and Combotox resulted in a survival advantage compared with saline-injected controls (p<0.001). Significantly, no improved survival could be seen when Ara-C and high-dose Combotox were given concurrently (Table I and FIG. 3C).

### Discussion

The results demonstrate that sequential administration of the cytotoxic agent cytarabine and the immunotoxin Combotox has synergistic anti-leukemic activity in an in vivo model of advanced B-lineage ALL. On direct comparison, sequential administration of both drugs at doses calculated to simulate human maximum tolerated doses resulted in increased survival when compared to concurrent administration.

These findings provide evidence that sequential chemoimmunotherapy results in superior outcomes when compared to cytotoxic therapy alone. For many hematologic malignancies, combining cytotoxic agents with immunotherapy has become the standard of care. However, the synergy seen herein is significant. The most notable example is the successful addition of the CD20-directed antibody rituximab to various chemotherapeutic backbones in indolent and aggressive CD20-positive B-cell malignancies, including mature B-cell ALL [17-22].

Another antigen, CD19, is present on virtually all malignant lymphoblasts in patients with B-lineage ALL, whereas the CD22 epitope is expressed on 80% of the blast population [23]. This makes these antigens excellent targets for immunotherapy in B-cell ALL. Both in vitro and in vivo experiments provide evidence for the activity of CD19 and CD22 antibody-drug conjugates against B-lineage leukemic lymphoblasts [14, 16,24-28]. The combination of certain drugs and agents targeting CD19 and CD22 was shown in in vitro experiments and xenograft models of early ALL to result in synergy [16,29].

Whereas these earlier experiments attempted to recapitulate stages of ALL with less disease burden by treating xenografted mice with antileukemic agents only 24 h after inoculation with the malignant cell line NALM/6 [16,25,27], the goal in the current experiment was to mimic an advanced leukemic phenotype and determine the effects on that condition. The synergy seen here was significant, and unexpectedly dependent on dosing schedule. Ara-C and/or Combotox was administered only when advanced disease with peripheral blasts was established. At this time leukemic blasts were readily detectable in the peripheral blood. Therefore the leukemic burden in the current model was much higher, and the experiment more reliably recapitulates the clinical picture encountered with human relapsed disease.

The observation that single-agent administration of only Ara-C or Combotox showed no significant survival difference in advanced disease as compared to placebo is noteworthy. This is in line with the generally accepted fact that, in patients with advanced ALL, single-agent therapy is often insufficient: outcomes are disappointing and the multi-agent approach needed to control this aggressive malignancy represents the current accepted standard of care [30-35]. However, the concurrent versus sequential dosing differences were not previously known, and the combination of cytarabine and Combotox is novel, and has been explored for the first time in this study. The antileukemic effect of the administered agents was assessed on the basis of median survival time. The xenografted mice could have died of either uncontrolled leukemia or toxicity of the administered drugs. As expected, none of the animals exhibited any visible signs of vascular leak syndrome, which is the most severe toxicity of Combotox, thus indicating that they died of disease progression. It is also reassuring that mice consistently survived longer when treated with chemoimmunotherapy in all three experiments. This shows a powerful anti-leukemic effect from the combination of cytarabine and Combotox, which resulted in longer survival. On the other hand, the observation that concurrent administration of high-dose Ara-C and Combotox resulted in survival similar to that of placebo-treated mice suggests that the significant toxicity of the concomitant therapy could have diminshed the antileukemic effect at these dose levels.

In summary, chemoimmunotherapy with Ara-C and Combotox synergistically improved survival when given sequentially in a murine model of advanced B-lineage ALL.

### REFERENCES

[1] Fielding A K, Richards S M, Chopra R, et al. Outcome of 609 adults after relapse of acute lymphoblastic leukemia (ALL); an MRC UKALL12/ECOG 2993 study. Blood 2007; 109:944-950.
[2] Faderl S, O′Brien S, Pui C-H, et al. Adult acute lymphoblastic leukemia. Cancer 2010; 116:1165-1176.
[3] FitzGerald D J, Wayne A S, Kreitman R J, et al. Treatment of hematologic malignancies with immunotoxins and antibody-drug conjugates. Cancer Res 2011; 71:6300-6309.
[4] Schindler J, Sausville E, Messmann R, et al. The toxicity of deglycosylated ricin A chain-containing immunotoxins in patients with non-Hodgkin's lymphoma is exacerbated by prior radiotherapy: a retrospective analysis of patients in five clinical trials. Clin Cancer Res 2001; 7:255-258.
[5] Messmann R A, Vitetta E S, Headlee D, et al. A phase I study of combination therapy with immunotoxins IgG-HD37-deglycosylated ricin A chain (dgA) and IgG-RFB4-dgA (Combotox) in patients with refractory CD19(+), CD22(+) B cell lymphoma. Clin Cancer Res 2000; 6:1302-1313 .
[6] Jegalian A G, Wayne A S, Kreitman R J, et al. CD22 expression in pediatric B-lineage acute lymphoblastic leukemia. Blood 2009; 114(Suppl. 1): Abstract 4119.

[7] May R D, Vitetta E S, Moldenhauer G, et al. Selective killing of normal and neoplastic human B cells with anti-CD19- and anti-CD22-ricin A chain immunotoxins. Cancer Drug Deliv 1986; 3:261-272.
[8] Herrera L, Bostrom B, Gore L, et al. A phase 1 study of Combotox in pediatric patients with refractory B-lineage acute lymphoblastic leukemia. J Pediatr Hematol Oncol 2009; 31:936-941.
[9] Topp M S, Zugmaier G, Goekbuget N, et al. Report of a phase II trial of single-agent BiTE(R) antibody blinatumomab in patients with minimal residual disease (MRD) positive B-precursor acute lymphoblastic leukemia (ALL). Blood 2009; 114(Suppl.1): Abstract 840.
[10] Topp M S, Kufer P, Gökbuget N, et al. Targeted therapy with the T-cell-engaging antibody blinatumomab of chemotherapy-refractory minimal residual disease in B-lineage acute lymphoblastic leukemia patients results in high response rate and prolonged leukemia-free survival. J Clin. Oncol 2011; 29:2493-2498.
[11] Dominietto A, Raiola A M, Van Lint M T, et al. Factors influencing haematological recovery after allogeneic haemopoietic stem cell transplants: graft-versus-host disease, donor type, cytomegalovirus infections and cell dose. Br J Haematol 2001; 112:219-227.
[12] Jabbour E, O′Brien S, Thomas D, et al. Inotuzumab ozogamicin (10; CMC544), a CD22 monoclonal antibody attached to calicheamycin, produces complete response (CR) plus complete marrow response (mCR) of greater than 50% in refractory relapse (R-R) acute lymphocytic leukemia (ALL). J Clin Oncol 2011; 29 (Suppl.): Abstract 6507.
[13] Schindler J, Gajavelli S, Ravandi F, et al. A phase I study of a combination of anti-CD19 and anti-CD22 immunotoxins (Combotox) in adult patients with refractory B-lineage acute lymphoblastic leukaemia. Br J Haematol 2011; 154:471-476.
[14] Herrera L, Farah R A, Pellegrini V A, et al. Immunotoxins against CD 19 and CD22 are effective in killing precursor-B acute lymphoblastic leukemia cells in vitro. Leukemia 2000; 14:853-858.
[15] Ghetie M A, Podar E M, Gordon B E, et al. Combination immunotoxin treatment and chemotherapy in SCID mice with advanced, disseminated Daudi lymphoma. Int J Cancer 1996; 68:93-96.
[16] Herrera L, Stanciu-Herrera C, Morgan C, et al. Anti-CD19 immunotoxin enhances the activity of chemotherapy in severe combined immunodeficient mice with human pre-B acute lymphoblastic leukemia. Leuk Lymphoma 2006; 47:2380-2387.
[17] Griffin T C, Weitzman S, Weinstein H, et al. A study of rituximab and ifosfamide, carboplatin, and etoposide chemotherapy in children with recurrent/refractory B-cell (CD20+) non-Hodgkin lymphoma and mature B-cell acute lymphoblastic leukemia: a report from the Children's Oncology Group. Pediatr Blood Cancer 2009; 52:177-181.
[18] Coiffier B, Lepage E, Briere J, et al. CHOP chemotherapy plus rituximab compared with CHOP alone in elderly patients with diffuse large-B-cell lymphoma. N Engl J Med 2002; 346:235-242.
[19] Pfreundschuh M, Trumper L, Osterborg A, et al. CHOP-like chemotherapy plus rituximab versus CHOP-like chemotherapy alone in young patients with good-prognosis diffuse large-B-cell lymphoma: a randomised controlled trial by the MabThera International Trial (MInT) Group. Lancet Oncol 2006; 7:379-391.
[20] Oriol A, Ribera J M, Bergua J, et al. High-dose chemotherapy and immunotherapy in adult Burkitt lymphoma: comparison of results in human immunodeficiency virus-infected and noninfected patients. Cancer 2008; 113:117-125.
[21] Corazzelli G, Frigeri F, Russo F, et al. RD-CODOX-M/IVAC with rituximab and intrathecal liposomal cytarabine in adult Burkitt lymphoma and “unclassifiable” highly aggressive B-cell lymphoma. Br J HaematoI2012; 156:234-244.
[22] Thomas D A, Faderl S, O′Brien S, et al. Chemoimmunotherapy with hyper-CVAD plus rituximab for the treatment of adult Burkitt and Burkitt-type lymphoma or acute lymphoblastic leukemia. Cancer 2006; 1 06: 1569-1580.
[23] Raponi S, Stefania De Propris M, Intoppa S, et al. Flow cytometric study of potential target antigens (CD19, CD20, CD22, CD33) for antibody-based immunotherapy in acute lymphoblastic leukemia: analysis of 552 cases. Leuk Lymphoma 2011; 52:1098-1107.
[24] Ghetie M-A, May R D, Till M, et al. Evaluation of ricin A chaincontaining immunotoxins directed against CD19 and CD22 antigens on normal and malignant human B-cells as potential reagents for in vivo therapy. Cancer Res 1988; 48:2610-2617.
[25] Herrera L, Yarbrough S, Ghetie V, et al. Treatment of SCID/human B cell precursor ALL with anti-CD19 and anti-CD22 immunotoxins. Leukemia 2003; 17:334-338.
[26] de Vries J F, Zwaan C M, De Bie M, et al. The novel calicheamicin-conjugated CD22 antibody inotuzumab ozogamicin (CMC-544) effectively kills primary pediatric acute lymphoblastic leukemia cells. Leukemia 2012; 26:255-264.
[27] Liu X-Y, Pop L M, Tsai L, et al. Chimeric, divalent and tetravalent anti-CD19 monoclonal antibodies with potent in vitro and in vivo antitumor activity against human B-cell-lymphoma and pre-B acute lymphoblastic leukemia cell lines. Int. J Cancer 2011; 129:497-506.
[28] Dreier T, Baeuerle P A, Fichtner I, et al. T cell costimulusindependent and very efficacious inhibition of tumor growth in mice bearing subcutaneous of leukemic human B cell lymphoma xenografts by a CD 19-/CD3-bispecific single-chain antibody construct. J Immunol 2003; 170:4397-4402.
[29] Stanciu-Herrera C, Morgan C, Herrera L. Anti-CD19 and anti-CD22 monoclonal antibodies increase the effectiveness of chemotherapy in pre-B acute lymphoblastic leukemia cell lines. Leuk Res 2008; 32:625-632.
[30] Advani A, Gundacker H, Sala-Torra O, et al. Southwest Oncology Group study 50530: a phase 2 trial of clofarabine/cytarabine for relapsed/refractory acute lymphocytic leukemia. Blood 2009; 114(Suppl 1): Abstract 3094.
[31] Larson R A, Dodge R K, Linker C A, et al. A randomized controlled trial of filgrastim during remission induction and consolidation chemotherapy for adults with acute lymphoblastic leukemia: CALGB study 9111. Blood 1998; 92:1550-1504.
[32] Ludwig W-D, Rieder H, Bartram C R, et al. Immunophenotypic and genotypic features, clinical characteristics, and treatment outcome of adult pro-B acute lymphoblastic leukemia: results of the German multicenter trials GMALL 03/87 and 04/89. Blood 1998; 92:1898-1909.
[33] Rowe J M, Buck G, Burnett A K, et al. Induction therapy for adults with acute lymphoblastic leukemia: results of more than 1500 patients from the international ALL trial: MRC UKALL XII/ECOG E2993. Blood 2005; 106:3760-3767.
[34] Ribera J, Oriol A, Bethencourt C, et al. Comparison of intensive chemotherapy, allogeneic or autologous stem cell transplantation as post-remission treatment for adult patients with high-risk acute lymphoblastic leukemia. Results of the PETHEMA ALL-93 trial. Haematologica 2005; 90:1346-1356.
[35] Bassan R, Hoelzer D. Modem therapy of acute lymphoblastic leukemia. J Clin Oncol 2011; 29:532-543.

What is claimed is:

1. A method of treating a leukemia in a mammal comprising sequentially administering to the mammal an amount of an immunotoxin and an amount of a chemotherapeutic agent effective to treat a leukemia.

2. The method of claim 1, wherein the mammal is a human.

3. The method of claim 1 or 2, wherein the leukemia is relapsed acute leukemia or refractory acute lymphoblastic leukemia.

4. The method of claim 1,2, or 3, wherein the immunotoxin comprises a combination of an anti-CD19 antibody or CD19-binding fragment thereof and an anti-CD22 antibody or CD22-binding fragment thereof

5. The method of any of claims 1-4, wherein the chemotherapeutic agent interferes with DNA synthesis.

6. The method of any of claims 1-5, wherein the chemotherapeutic agent is cytarabine.

7. The method of claim 6, wherein the mammal is a human and the cytarabine is administered at a dosage of 50-3000 milligrams per square meter of the human's skin area.

8. The method of any of claims 1-5, wherein the amount of chemotherapeutic agent administered is 100 to 300 milligrams per kilogram of body weight of the mammal.

9. The method of any of claims 1-5, wherein the amount of chemotherapeutic agent administered is 100 to 300 milligrams per square meter of the mammal's skin area.

10. The method of any of claims 1-9, wherein the amount of immunotoxin comprises 1 to 4 milligrams of anti-CD19 antibody per kilogram of body weight of the mammal and 0.5 to 2.5 milligrams of anti-CD22 antibody per kilogram of body weight of the mammal.

11. The method of any of claims 1-9, wherein the amount of immunotoxin comprises
0.5 to 1.5 milligrams of anti-CD19 antibody per kilogram of body weight of the mammal and
0.1 to 1.0 milligrams of anti-CD22 antibody per kilogram of body weight of the mammal.

12. The method of any of claims 1-11, wherein the immunotoxin is administered before the chemotherapeutic agent is administered.

13. The method of any of claims 1-11, wherein the chemotherapeutic agent is administered before the immunotoxin is administered.

14. The method of any of claims 1-11, wherein the sequential administration comprises treatment with the chemotherapeutic agent on three consecutive days;
four subsequent days of no administration of the chemotherapeutic agent or the immunotoxin;
treatment with immunotoxin on the next subsequent day;
one subsequent day of no administration of the chemotherapeutic agent or the immunotoxin;
treatment with immunotoxin on the next subsequent day;
one subsequent day of no administration of the chemotherapeutic agent or the immunotoxin; and
treatment with immunotoxin on the next subsequent day.

15. The method of any of claims 1-11, wherein the sequential administration comprises administration of the chemotherapeutic and subsequent administration of the immunotoxin.

16. The method of any of claims 1-11, wherein the sequential administration comprises administration of the immunotoxin and subsequent administration of the chemotherapeutic.

17. The method of claim 15 or 16, wherein the immunotoxin is not administered within one of 1 hr., 2 hrs., 4 hrs., 8 hrs., 24 hrs or 48 hrs. of the administration of the chemotherapeutic.

18. The method of any of claims 1-14, wherein the immunotoxin comprises anti-CD19 antibody coupled to a deglycosylated ricin-A chain and/or anti-CD22 antibody coupled to a deglycosylated ricin-A chain.

19. The method of any of claims 4-15, wherein the anti-CD19 and anti-CD22 antibodies are humanized antibodies.

20. The method of any of claims 1-19, wherein the leukemia is advanced and is characterized by the presence of peripheral blasts in the mammal.
